(19) United States
(12) Patent Application Publication
(10) Pub. No.: US 2019/0151256 A1
(43) Pub. Date: May 23, 2019

(54) Composition Including Nonanal As Active Ingredient For Preventing Hair Loss Or Stimulating Hair Development

(71) Applicant: Industry-Academic Cooperation Foundation, Yonsei University, Seoul (KR)

(72) Inventor: Taesun PARK, Seoul (KR)

(73) Assignee: Industry-Academic Cooperation Foundation, Yonsei University, Seoul (KR)

(21) Appl. No.: 16/171,274

(22) Filed: Oct. 25, 2018

(30) Foreign Application Priority Data
Oct. 25, 2017 (KR) 10-2017-0139172

(51) Int. Cl. A61K 31/11; A61K 8/33; A61P 17/14

(52) U.S. Cl. CPC A61K 31/11; A61K 9/0014; A61P 17/14; A61K 8/33

(57) ABSTRACT

The present invention relates to a composition including nonanal or a pharmaceutically acceptable salt thereof as an active ingredient for preventing or treating hair loss or promoting hair development, and more particularly, to a pharmaceutical composition, a quasi-drug composition, a cosmetic composition and a health functional food composition, which include nonanal or a pharmaceutically acceptable salt thereof as an active ingredient for preventing or treating hair loss or promoting hair development or growth. The composition of the present invention may include a natural compound as an active ingredient, have no side effects during long-term administration for treating hair loss, which is a chronic disease, and exhibit excellent and stable efficacy in hair development and hair growth, and therefore it can be effectively used as compositions for an agent for preventing or treating hair loss, and a drug a quasi-drug, a cosmetic, or health functional food for promoting hair development or growth.

### CROSS-REFERENCE TO RELATED APPLICATION

This application claims priority to and the benefit of Korean Patent Application No. 10-2017-0139172, filed on Oct. 25, 2017, the disclosure of which is incorporated herein by reference in its entirety.

### BACKGROUND

### 1. Field of the Invention

The present invention relates to a composition including nonanal or a pharmaceutically acceptable salt thereof as an active ingredient for preventing hair loss or stimulating hair development, and more particularly, to a pharmaceutical composition, a quasi-drug composition, a cosmetic composition, and a health functional food composition, which include nonanal or a salt thereof as an active ingredient for preventing or treating hair loss or stimulating hair development and growth.

### 2. Discussion of Related Art

According to analysis of the health insurance payment data from 2001 to 2008, conducted by the Health Insurance Policy Research Institute in the National Health Insurance Corporation, the number of patients treated for “alopecia” increased from 103,000 in 2001 to 142,000 in 2005, and 165,000 in 2008, increasing by 60% over the seven years. According to age, the number of treated patients aged 20 to 40 years was 114,000, accounting for 69.5%, and the number of treated patients in their teens or lower was 22,000 or more. According to gender, the numbers of treated patients were 84,000 males and 80,000 females in 2008, showing that the males are slightly more than females, and according to type of “alopecia”, the numbers of treated patients with health care coverage in Korea were 130,000 (prototype alopecia), 20,000 (scarring alopecia), 9,000 (androgenic alopecia), and 8,000 (other non-scarring hair loss) in 2008.

Meanwhile, according to the International Hair and Cosmetic Workshop, held in June, 2003, 250 million people around the world were hair loss patients, and the incidence of hair loss in patients aged 24 to 50 was approximately 30 to 65%. In 2008, China's hair loss population was approximately 300 million, approximately 30% of the male population in their 30s and approximately 50% of the male population in their 50s had hair loss symptoms, and the number of hair loss patients is increasing by approximately 10 to 15% each year. In 2007, according to a survey of companies that sell wigs and perform hair transplantation surgery targeting Japanese people, the incidence rate of hair loss was 26.5%, and the number of people with hair loss was estimated to be approximately 12.93 million.

Currently, preparations for treating hair loss are largely classified as medicines, quasi-drugs, and cosmetics. As a commercially available prescription medicine, there is “Propecia” which is developed and distributed by Merck & Co., Inc. Finasteride, which is the main component of Propecia, has been approved as a medication for treating hair loss by the US FDA in December, 1997. Finasteride is a medication for inhibiting 5-α-reductase that converts testosterone into dihydrotestosterone (DHT) and acts to make hair thicker and longer. Finasteride has an effect of improving hair loss in the short term, but is accompanied by side effects such as erectile dysfunction, sexual dysfunction, male breast enlargement, etc.

As an over-the-counter medication, Minoxidil, which has been proven to have stability and efficacy, can be obtained without a doctor's prescription, and has been approved as the first applicable drug for treating hair loss by the US FDA in December 1997. This drug is effective in promoting blood circulation and opening a potassium channel, resulting in the promotion of hair growth, but may lead to local reactions such as itching, and a rash, and tachycardia.

Among quasi-drugs which can be purchased from a supermarket or convenience store according to a notification of the Ministry of Health and Welfare as products approved by the KFDA and products proven to be effective in prevention of hair loss and hair growth, and as representative examples, there are “Moballyeok Competent” produced by CJ Lion, “Hair Tonic” produced by Moracle, and “MO & MORE” produced by LG Household & Health Care Ltd., and cosmetics, shampoos or products used for the scalp or hair for maintaining or improving the health of skin and hair are being marketed.

A human's hair development cycle is largely classified into an anagen phase, catagen phase, and telogen phase. The anagen phase is a period in which hair follicles are nourished and cell division actively occurs, thereby growing hair fast. The lifespan of hair in the anagen phase varies according to the type of hair, but the lifespan of human hair is approximately 3 to 6 years. The anagen-phase hair accounts for 80 to 90% of the entire hair, and people who are undergoing hair loss tend to have a hair cycle with a shorter anagen phase and a longer telogen phase, thereby decreasing the proportion of the anagen-phase hair among the entire hair. The catagen phase is a period in which the anagen phase of hair ends and hair regeneration gradually slows down, and then cell division and growth stop and lasts approximately 1 to 1.5 months, and approximately 1% of the entire hair is in this phase. The telogen phase is the last stage of growth, and a period in which the hair follicle and the dermal papilla are completely separated, and thus the hair follicle is shrunken and the hair follicle goes up, thereby shedding hair. The telogen phase lasts 3 to 4 months, and 4 to 14% of entire hair is in this phase. When the telogen phase ends and the dermal papilla are actively stimulated again, a new hair follicle is made and thus the telogen hair is pushed out and completely released from the scalp.

However, nonanal is a component of essential oils derived from various plants, and has been known as a safe and edible material. Nonanal was approved as a component of flavoring and fragrance agents by the Flavor and Extract Manufacturers' Association (FEMA), Food and Drug Administration (FDA), Korea Food and Drug Administration (KFDA), Council of Europe (COE), and Joint FAO/WHO Expert Committee on Food Additives (JECFA). In addition, nonanal has been used as a flavoring and fragrance agent to flavor geranium, rose or tea to produce a perfume, endow foods with a waxy, aldehydic, rose, fresh orris, orange peel, cucumber, citrus, melon, potato, or coconut flavor, and add sweetness to a citrus flavor. However, the effect of nonanal on preventing hair loss or promoting hair development is not yet been known.

### SUMMARY OF THE INVENTION

Therefore, the inventors had attempted to discover a natural compound which can prevent hair loss or promote hair development or growth without side effects. As a result, they had confirmed the fact that nonanal effectively promotes hair development and growth and prevents hair loss, and thus the present invention was completed.

Therefore, to solve the above-mentioned problem, an object of the present invention provides a pharmaceutical composition including nonanal or a pharmaceutically acceptable salt thereof as an active ingredient for preventing or treating hair loss, or promoting hair development or growth.

Another object of the present invention provides a quasi-drug composition including nonanal or a pharmaceutically acceptable salt thereof as an active ingredient for preventing or improving hair loss, or promoting hair development or growth.

Still another object of the present invention provides a cosmetic composition including nonanal or a cosmetologically acceptable salt thereof as an active ingredient for preventing or improving hair loss, or promoting hair development or growth.

Yet another object of the present invention provides a health functional food composition including nonanal or a sitologically acceptable salt thereof as an active ingredient for preventing or improving hair loss, or promoting hair development or growth.

However, technical problems to be solved in the present invention are not limited to the above-described problems, and other problems which are not described herein will be fully understood by those of ordinary skill in the art from the following descriptions.

Therefore, the present invention provides a pharmaceutical composition including nonanal or a pharmaceutically acceptable salt thereof as an active ingredient for preventing or treating hair loss, or promoting hair development or growth.

According to an exemplary embodiment of the present invention, the composition may increase the expression of a protein selected from the group consisting of β-catenin, a T-cell factor (TCF), a lymphoid enhancer factor (LEF) and cyclin D1.

According to another exemplary embodiment of the present invention, the pharmaceutical composition may be prepared in the form selected from the group consisting of a cream, a gel, a patch, a spray, an ointment, a plaster, a lotion, a liniment, a paste, and a cataplasm as a drug for topical use.

The present invention also provides a quasi-drug composition including nonanal or a pharmaceutically acceptable salt thereof as an active ingredient for preventing or improving hair loss, or promoting hair development or growth.

The present invention also provides a cosmetic composition including nonanal or a cosmetologically acceptable salt thereof as an active ingredient for preventing or improving hair loss, or promoting hair development or growth.

According to still another exemplary embodiment of the present invention, the cosmetic composition may be prepared in the form of a scalp tonic, a scalp lotion, a scalp cream, a scalp serum, a scalp essence, a scalp ampoule, a scalp treatment, a scalp conditioner, a scalp shampoo, a scalp pack, a hair tonic, a hair lotion, a hair cream, a hair spray, a hair mousse, a hair gel, a hair conditioner, a hair shampoo, a hair rinse, a hair pack, a hair treatment, an eyebrow growth serum, an eyelash growth serum, an eyelash enhancer, and shampoos and rinses for pets.

The present invention also provides a health functional food composition including nonanal or a pharmaceutically acceptable salt thereof as an active ingredient for preventing or improving hair loss, or promoting hair development.

According to an aspect of another embodiment, there is provided a method for preventing or treating hair loss or promoting hair development or growth, the method comprising administering a composition including nonanal or a pharmaceutically acceptable salt thereof as an active ingredient, applying it or allowing it to be taken.

According to another exemplary embodiment of the present invention, the composition is pharmaceutical composition, quasi-drug composition, cosmetic composition or health functional food composition.

The present invention has been suggested to solve the above-mentioned problems, and is directed to providing a composition including nonanal or a pharmaceutically acceptable salt thereof for preventing or improving hair loss, or promoting hair development or growth.

The composition of the present invention includes a natural compound as an active ingredient which is effective against hair loss, which is a chronic disease, without side effects even in long-term administration, exhibits excellent and stable efficacy in hair development and growth, and thus can be effectively used as a drug for preventing or treating hair loss, or a quasi-drug, cosmetic or health functional food composition for promoting hair development.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a set of images showing hair restoration in shaved mouse models according to days of administering each sample;

FIG. 2 is a graph showing hair lengths grown while each sample is applied to shaved mouse models. Respective values represent mean±standard deviations in five mice. ns=p>0.05, **=p<0.01, ***=p<0.001 [one-way ANOVA, Tukey's test];

FIG. 3 is a set of graphs obtained by histological observation of mice to which each sample was applied for four weeks. FIG. 3A shows the result of confirming an increase in the number of hair follicles, and FIG. 3B shows the result of measuring the diameters of the hair follicles. Respective values represent mean±standard deviations in five mice. ns=p>0.05, **=p<0.01, ***=p<0.001 [one-way ANOVA, Tukey's test];

FIG. 4 is a set of optical microscope images showing that hair follicles are grown out of the skin of mice to which each sample is applied for 4 weeks;

FIG. 5 shows changes in β-catenin protein expression of skin tissue of a mouse to which each sample is applied. Western blotting results are mean±standard deviations for independent experiments in each group (for each experiment, n=2 or 3). ns=p>0.05, **=p<0.01 [one-way ANOVA, Tukey's test]; and

FIG. 6 shows changes in gene expression of skin tissue of a mouse to which each sample is applied. Western blotting results are mean±standard deviations for independent experiments in each group (for each experiment, n=2 or 3). ns=p>0.05, *=p<0.05, **=p<0.01, ***=p<0.001 [one-way ANOVA, Tukey's test].

### DETAILED DESCRIPTION OF EXEMPLARY EMBODIMENTS

Hereinafter, the present invention will be described in detail.

As described above, as a hair loss phenomenon increases due to stress in addition to a change in hormone secretion, there is an increasing demand for a pharmaceutical composition which can prevent hair loss and/or promote hair development. However, among natural compounds known to be safe to a human body, a compound that can effectively prevent hair loss and/or promote hair development has not been sufficiently reported.

Since the nonanal of the present invention can significantly exhibit an effect of promoting hair development in hair-removed mice by adjusting expression of a hair development-related gene and a protein of skin tissue, a composition effective in preventing hair loss and promoting hair growth is provided.

Therefore, the present invention provides a pharmaceutical composition including nonanal or a pharmaceutically acceptable salt thereof as an active ingredient for preventing or treating hair loss or promoting hair development or growth.

The term “nonanal” used herein is an alkyl aldehyde-based compound represented by Formula 1 below, and is called nonanal in accordance with its IUPAC name and is also called pelargonaldehyde, nonanaldehyde, nonyl aldehyde or C-9 aldehyde. The structural formula of nonanal is C₉H₁₈O, and its molecular weight is 142 g/mol. Nonanal is a colorless liquid, and well dissolved in an alcohol or propylene glycol.

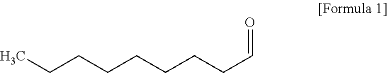

Nonanal is a component of essential oils obtained from various plants, has been known as a safe and edible material, and is industrially used to endow food with a flavor and fragrance. According to oral toxicity test results, it was shown that the lethal dose 50% (LD₅₀ value) of nonanal was 5,000 mg/kg in rats. Meanwhile, according to the result of a transdermal toxicity test for rabbits, the LD₅₀ value was 5,000 mg/kg. In addition, as a result of clinical studies, it has been reported that there was no dermal irritation or sensitization reaction (sensitivity) in a 1% solution.

Meanwhile, in experiments with mice, it was reported that nonanal exhibits antidiarrheal activity. As a result of evaluating the effect of castor oil on intestinal passage in diarrhea-induced mice using magnesium sulfate, arachidonic acid or a PGE2 complex, it has been reported that, among components of essential oils of castor oil, nonanal exhibits a significant effect in inhibition of diarrhea, and diarrhea does not occur (Zavala-Sanchez et al., Antidiarrheal activity of nonanal, an aldehyde isolated from Artemisia ludoviciana, Pharmaceutical Biology, 40(4); 263-268, 2002).

Accordingly, when nonanal is administrated orally at 0.1 mg/kg to 1,500 mg/kg, and preferably 10 mg/kg to 1,000 mg/kg, it exhibits little or no toxicity, and therefore, nonanal can be used in a cosmetic composition, a health functional food composition, a medication or a quasi-drug composition.

In the present invention, the nonanal may include a nonanal hydrate or nonanal derivative, or a solvate or stereoisomer thereof within a range having the same efficacy as described above.

In the present invention, by identifying a mechanism of regulating a hair development-related gene and protein expression of skin tissue, a novel use of nonanal for preventing hair loss or promoting hair development was confirmed.

The term “hair loss” used herein refers to a phenomenon of completely losing hair from the scalp. A person losing hair has a hair growth cycle with a short anagen phase and a long telogen phase, and as to be proved in the following Examples, it can be confirmed that nonanal allows hair to be converted from a telogen phase to an anagen phase, and increases expression of a hair development-related growth factor of skin tissue, resulting in an effect of preventing, improving or treating hair loss.

The term “hair development” used herein refers to generation of hair on the scalp, and the term “hair growth” refers to elongation of hair (that is, the growth of hair) and is used interchangeably with another term “hair thickening” used in in the art.

The composition of the present invention may increase expression of a protein selected from the group consisting of β-catenin, TCF, LEF and cyclin D1. Specifically, as shown in Example 2 of the present invention, it was confirmed that the composition of the present invention increases expression of a β-catenin gene related with a Wnt/β-catenin signaling system involved in causing the cycle of hair follicles in skin tissue to enter an anagen phase, and expression of all of β-catenin, downstream signaling transcription factors TCF and LEF, and a target gene cyclin D1, which has been known as a mechanism of promoting hair development, is significantly increased. In addition, currently, it was confirmed that an expression level of the protein or gene is significantly increased, compared with a group treated with Minoxidil, which is a representative drug that can promote hair development and growth. Therefore, the composition of the present invention is less harmful to the human body than a conventional synthetic composition, and can be effectively used as a hair development or growth composition exhibiting various and stable effects of promoting hair development and growth.

As the nonanal is preferably a composition for a drug for topical use, a pharmaceutical composition for topical use, which includes the nonanal of the present invention as an active ingredient, for preventing hair loss and improving and promoting hair development may be prepared in the form of a cream, a gel, a patch, a spray, an ointment, a plaster, a lotion, a liniment, a paste, and a cataplasm, but the present invention is not limited thereto.

The nonanal of the present invention may be used in the form of a pharmaceutically acceptable salt, and as the salt, an acid-addition salt formed by a pharmaceutically acceptable free acid is useful. The acid-addition salt is obtained from inorganic acids such as hydrochloric acid, nitric acid, phosphoric acid, sulfuric acid, hydrobromic acid, hydroiodic acid, nitrous acid and phosphorous acid, and non-toxic organic acids such as aliphatic mono and dicarboxylates, phenyl-substituted alkanoates, hydroxyalkanoates and alkanedioates, aromatic acids, aliphatic and aromatic sulfonic acids. The pharmaceutically non-toxic salts include sulfates, pyrosulfates, bisulfates, sulfites, bisulfites, nitrates, phosphates, monohydrogen phosphates, dihydrogen phosphates, metaphosphates, pyrophosphate chlorides, bromides, iodides, fluorides, acetates, propionates, decanoates, caprylates, acrylates, formates, isobutyrates, caprates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, terephthalates, benzene sulfonates, toluene sulfonates, chlorobenzene sulfonates, xylene sulfonates, phenylacetates, phenylpropionates, phenylbutyrates, citrates, lactates, β-hydroxybutyrates, glycolates, malates, tartrates, methanesulfonates, propanesulfonates, naphthalene-1-sulfonates, naphthalene-2-sulfonates, and mandelates.

The acid-addition salt according to the present invention may be prepared by a conventional method, for example, by dissolving the nonanal in an excessive amount of an aqueous acid solution, and precipitating the salt using a water-miscible organic solvent, for example, methanol, ethanol, acetone or acetonitrile. The acid-addition salt according to the present invention may be prepared by heating an equivalent amount of the nonanal and an acid in water or an alcohol, and then suction-filtering a salt dried or precipitated by evaporating the mixture.

In addition, a pharmaceutically acceptable metal salt may be prepared using a base. The alkali metal or alkaline earth metal salt is obtained, for example, by dissolving a compound in an excessive amount of an alkali metal oxide or alkali earth metal hydroxide solution, filtering a non-dissolved compound salt, and evaporating a filtrate to dryness. At this time, as a metal salt, a sodium, potassium or calcium salt is pharmaceutically suitable. In addition, a silver salt corresponding thereto is obtained by reacting an alkali metal or alkali earth metal salt with a suitable silver salt (e.g., silver nitrate).

In addition, the nonanal of the present invention includes all types of salts, hydrates, and solvates, which can be prepared by a conventional method, as well as pharmaceutically acceptable salts.

The addition salt may be prepared by a conventional method, and may be prepared, for example, by dissolving nonanal in a water-miscible organic solvent, for example, acetone, methanol, ethanol or acetonitrile, and performing precipitation or crystallization by adding an excessive amount of organic acid or an aqueous inorganic acid solution. Subsequently, a solvent or an excessive amount of acid may be evaporated from the mixture and dried, thereby obtaining the addition salt, or a precipitated salt may be prepared by suction filtration.

When the composition of the present invention is used as a medication, a pharmaceutical composition containing nonanal or a pharmaceutically acceptable salt as an active ingredient may be formulated into various oral or parenteral dosage forms upon clinical administration, but the present invention is not limited thereto.

Examples of preparations for oral administration may include a tablet, a pill, hard/soft capsules, a liquid, a suspension, an emulsion, a syrup, a granule, and an elixir, and may contain a diluent (e.g., lactose, dextrose, sucrose, mannitol, sorbitol, cellulose and/or glycine), a lubricant (e.g., silica, talc, stearic acid and a magnesium or calcium salt thereof, and/or polyethylene glycol), in addition to an active ingredient. In addition, tablets may contain a binder such as magnesium aluminum silicate, a starch paste, gelatin, methylcellulose, sodium carboxymethylcellulose and/or polyvinylpyrrolidine, and in some cases, may contain a disintegrant such as starch, agar, alginic acid or a sodium salt thereof, or a boiling mixture, and/or an absorbent, a coloring agent, a flavoring agent, and a sweetening agent.

The pharmaceutical composition containing the nonanal of the present invention or a pharmaceutically acceptable salt thereof as an active ingredient may be parenterally administered, and parenteral administration is carried out by subcutaneous injection, intravenous injection, intramuscular injection or intrathoracic injection. At this time, to form a preparation for parenteral administration, the nonanal or a pharmaceutically acceptable salt thereof is mixed with a stabilizer or buffer in water, thereby obtaining a solution or suspension, and then the solution or suspension may be prepared in the form of ampoules or vial-units. The composition may be sterilized and/or contain additives such as a preservative, a stabilizer, a solubilizer or emulsifier, a salt for regulating osmosis and/or a buffer, and other therapeutically effective materials, and may be prepared by a conventional method such as mixing, granulation or coating.

In addition, an administration dose of the composition of the present invention to the human body may vary according to a patient's age, body weight and gender, an administration route, a health condition, and the severity of a disease, and when based on an adult patient having a body weight of 60 kg, the composition of the present invention may be generally administered at 0.001 to 1,000 mg/day, preferably 0.01 to 500 mg/day, at regular intervals one to several times per day according to the determination of a doctor or pharmacist.

In addition, the present invention provides a quasi-drug including nonanal or a pharmaceutically acceptable salt thereof as an active ingredient for preventing or improving hair loss, or promoting hair development or growth. Details of the nonanal are as described above.

When the nonanal of the present invention is used as an active ingredient of the quasi-drug composition, the nonanal exhibiting effects of preventing hair loss and promoting hair growth may be added alone, or in combination with other quasi-drugs or quasi-drug components, and may be suitably used according to a conventional method. The mixing ratio of the active ingredients may be suitably determined according to the purpose of use.

There is no particular limitation to the form of the quasi-drug composition for preventing hair loss and promoting hair growth, and thus the quasi-drug composition may be formulated in various quasi-drug preparations, which are effective in preventing hair loss and promoting hair growth, known in the art. The preparations for a quasi-drug may include a scalp tonic, a scalp lotion, a scalp cream, a scalp serum, a scalp essence, a scalp ampoule, a scalp treatment, a scalp conditioner, a scalp shampoo, a scalp pack, a hair tonic, a hair lotion, a hair cream, a hair spray, a hair mousse, a hair gel, a hair conditioner, a hair shampoo, a hair rinse, a hair pack, a hair treatment, an eyebrow growth serum, an eyelash growth serum, an eyelash enhancer, and shampoos and rinses for pets, a hand sanitizer, a detergent, a soap, an antiseptic cleanser, a wet tissue, a mask, an ointment, a patch, or a filter filler, and all quasi-drugs in the usual sense are included.

In addition, for each preparation, the quasi-drug composition for preventing hair loss and promoting hair growth may contain different components, which may be arbitrarily selected according to a preparation or the purpose of use and mixed. The mixing ratio of active ingredients may be suitably determined according to the purpose of use, and may include, for example, conventional additives such as a thickening agent, a stabilizer, a solubilizer, a vitamin, a pigment, and a fragrance, and a carrier.

A content of the composition may be 0.0001 to 10 wt % based on the total weight, and when the content is more than 10 wt %, a color and stability of the composition are degraded when prepared, and when the content is less than 0.0001 wt %, the composition exhibits an insignificant effect. The quasi-drug composition including the nonanal as an active ingredient has almost no toxicity and side effects as confirmed from the therapeutic index, and thus can be effectively used as a material for quasi-drugs.

In addition, the present invention provides a cosmetic composition including nonanal or a pharmaceutically acceptable salt thereof as an active ingredient for preventing or improving hair loss or promoting hair development or growth.

Components contained in the cosmetic composition of the present invention include components conventionally used in a cosmetic composition, in addition to nonanal or a pharmaceutically acceptable salt thereof as an active ingredient, and contains conventional additives such as an antioxidant, a stabilizer, a solubilizer, a vitamin, a pigment and a fragrance, and a carrier.

The cosmetic composition of the present invention may be prepared in any form conventionally used in the art, for example, a solution, a suspension, an emulsion, a paste, a gel, a cream, a lotion, a powder, a soap, a surfactant-containing cleanser, an oil, a powder-type foundation, an emulsion-type foundation, a wax foundation or a spray, but the present invention is not limited thereto.

When the cosmetic composition of the present invention is prepared in the form of a paste, a cream or a gel, animal oil, vegetable oil, wax, paraffin, starch, tragacanth, a cellulose derivative, polyethylene glycol, silicon, bentonite, silica, talc or zinc oxide may be used as a carrier component.

When the cosmetic composition of the present invention is prepared in the form of a powder or spray, as a carrier component, lactose, talc, silica, aluminum hydroxide, calcium silicate or a polyamide powder may be used, and particularly, in the case of a spray, additionally, a propellant such as chlorofluorohydrocarbon, propane/butane or dimethyl ether may be contained.

When the cosmetic composition of the present invention is prepared in the form of a solution or emulsion, as a carrier component, a solvent, a solubilizer or an emulsifier is used. For example, water, ethanol, isopropanol, ethyl carbonate, ethyl acetate, benzyl alcohol, benzyl benzoate, propylene glycol, 1,3-butylglycol oil, glycerol aliphatic ester, polyethylene glycol or a sorbitan fatty acid ester may be used.

When the cosmetic composition of the present invention is prepared in the form of a suspension, as a carrier component, a liquid diluent such as water, ethanol or propylene glycol, a suspending agent such as ethoxylated isostearyl alcohol, a polyoxyethylene sorbitol ester or polyoxyethylene sorbitan ester, microcrystalline cellulose, aluminum metahydroxide, bentonite, agar or tragacanth may be used.

When the cosmetic composition of the present invention is prepared in the form of a surfactant-containing cleanser, as a carrier component, aliphatic alcohol sulfate, aliphatic alcohol ether sulfate, sulfosuccinic acid monoester, isethionate, an imidaxolinium derivative, methyltaurate, sarcosinate, fatty acid amide ether sulfate, alklyamidobetaine, an aliphatic alcohol, fatty acid glyceride, fatty acid diethanolamide, vegetable oil, a lanolin derivative, or an ethoxylated glycerol fatty acid ester may be used.

In addition, the present invention provides a health functional food composition including nonanal or a pharmaceutically acceptable salt thereof as an active ingredient preventing or improving hair loss or promoting hair development or growth.

The food composition according to the present invention may be prepared in various forms according to a conventional method known in the art. As a common food, the food composition may be prepared by adding the nonanal of the present invention to any one of beverages (including alcoholic beverages), fruits and processed foods (e.g., canned fruit, preserved fruit, jam, marmalade, etc.), fish, meat and processed food thereof (e.g., ham, sausage corn beef, etc.), bread and noodles (e.g., udon, soba, ramen, spaghetti, macaroni, etc.), fruit juice, various kinds of drinks, cookies, taffies, dairy products (e.g., butter, cheese, etc.), edible vegetable oil, margarine, vegetable proteins, retort foods, frozen foods, or a variety of condiments (e.g., soy bean paste, a soy bean sauce, a sauce, etc.), but the present invention is not limited thereto. In addition, as a nutritional supplement, the food composition may be prepared by adding the nonanal of the present invention to a capsule, tablet or pill, but the present invention is not limited thereto. In addition, as a food functional food, the food composition may be prepared by liquefying, granulating, encapsulating or pulverizing the nonanal of the present invention to be ingested as a tea, juice or drink, but the present invention is not limited thereto. In addition, the nonanal of the present invention may be prepared in the form of a powder or concentrate to be used as a food additive. In addition, the nonanal of the present invention may be mixed with an active ingredient conventionally known to be effective in preventing hair loss and promoting hair development, thereby preparing a composition.

When the nonanal of the present invention is used as a health beverage, the health beverage composition may contain various flavoring and fragrance agents or natural carbohydrates as additional ingredients, like a conventional beverage. The above-described natural carbohydrates may include monosaccharides such as glucose, fructose, etc.; disaccharides such as maltose, sucrose, etc.; polysaccharides such as dextrin, cyclodextrin, etc.; and sugar alcohols such as xylitol, sorbitol, erythritol, etc. The sweetening agent may be a natural sweetening agent such as thaumatin or a stevia extract; or a synthetic sweetening agent such as saccharin or aspartame. Generally, a proportion of the natural carbohydrate may be approximately 0.01 to 0.04 g, and preferably, approximately 0.02 to 0.03 g per 100 mL of the composition of the present invention.

In addition, the nonanal of the present invention may be contained as an active ingredient for health functional food for preventing hair loss and promoting hair development, and a content of the nonanal of the present invention is not particularly limited as long as it is effective in achieving effects of preventing hair loss and promoting hair growth, and is preferably 0.01 to 100 wt % with respect to the total weight of the composition. The food composition of the present invention may be prepared by mixing nonanal with other active ingredients which have been known to be effective in preventing hair loss and promoting hair growth.

In addition to the above-mentioned components, the health food of the present invention may contain various nutrients, vitamins, electrolytes, flavoring and fragrance agents, coloring agents, pectic acid, pectates, alginic acid, alginates, organic acids, a protective colloidal thickening agent, a pH adjustor, a stabilizer, a preservative, glycerin, an alcohol, or a carbonizing agent. In addition, the health food of the present invention may contain fruit flesh for preparing natural fruit juice, fruit juice-flavored drinks, or vegetable drinks. Such components may be used alone or in combination. Proportions of these additives are not very important, but are generally selected within a range of 0.01 to 0.1 part by weight with respect to 100 parts by weight of the composition of the present invention.

Hereinafter, the present disclosure will be described in detail with reference to examples. However, these examples are for further illustrating the present disclosure, and the scope of the present disclosure is not limited to these examples.

### EXAMPLES

### Example 1

### Efficacy of Nonanal on Promoting Hair Development

1-1. Breeding of Experimental Animals and Application of Agent for Hair Development

(1) Preparation of Sample

All of a vehicle (ethanol:water:propylene glycol=5:3:2), a control drug, Minoxidil (MXD), and a test material, nonanal (NN) were purchased from the Sigma-Aldrich Corp.

(2) Breeding of Experimental Animals

Experiments were carried out using 24 male C57BL/6N mice aged 6 weeks, which were purchased from OrientBio Inc., acclimated for two weeks in a laboratory environment, and then divided into a total of three groups (n=5) of a negative control (CON), a positive control (MXD) and a nonanal group (NN). An animal laboratory was maintained at 21±2.0° C., a relative humidity of 50±5%, on a 12-hour day/night cycle. During the experimental period, a general solid feed (chow) and water were freely offered to the mice.

(3) Dermal Application of Agent for Hair Development and Visual Observation Method

To investigate a hair development effect, an 8-week-old mouse with telogen-phase hair, in which a dorsal skin color was pink, were used. The dorsal hair of the mouse was removed using a clipper for a mouse, and the sample was applied daily at 4 p.m. for 4 weeks. The test material nonanal (3 mg/mouse/day) and the control drug Minoxidil (3 mg/mouse/day) were applied after being dissolved in the vehicle (ethanol:water:propylene glycol=5:3:2), and only the vehicle was applied to the negative control group.

To visually examine a hair growth state, 1, 2, 3, and 4 weeks after the application started, the experimental animals were slightly anesthetized with ether, and a dorsal region was photographed. To evaluate the degree of hair growth, the hair length was measured using a ruler.

1-2. Assessment of Change in Body Weight

A body weight of the experimental animal from immediately before the application of an agent for hair development to the end of the application was measured weekly. There was no significant difference in initial weight between the negative control group (CON group), the experimental group (NN group), and the positive control group (MXD group), and there was also no significant difference in body weight measured after 4 weeks of application of the test material between the test groups.

1-3. Hair Development State and Change in Hair Length

(1) Visual Characteristic of Hair Development

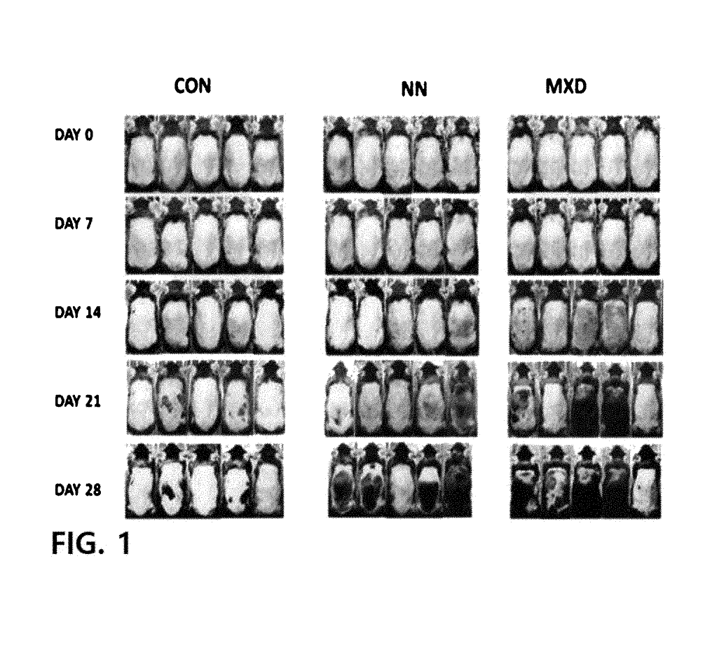

The specimen was applied to the dorsal region of the mouse for 4 weeks, and the aspect of hair growth was examined weekly by photographing. When shaved, the color of the body surface of a telogen-phase mouse was pink. When the color of the body surface turned black as the experiment progressed, it indicates that the hair cycle enters the anagen phase from the telogen phase. It was observed that, in the case of the negative control group (CON), after three weeks, there was almost no hair growth, whereas in the nonanal applied group (NN), from three weeks of the application, hair began to grow and after 4 weeks, hair grew more evenly and fully in more individuals than that in the negative control group (FIG. 1).

(2) Change in Hair Length

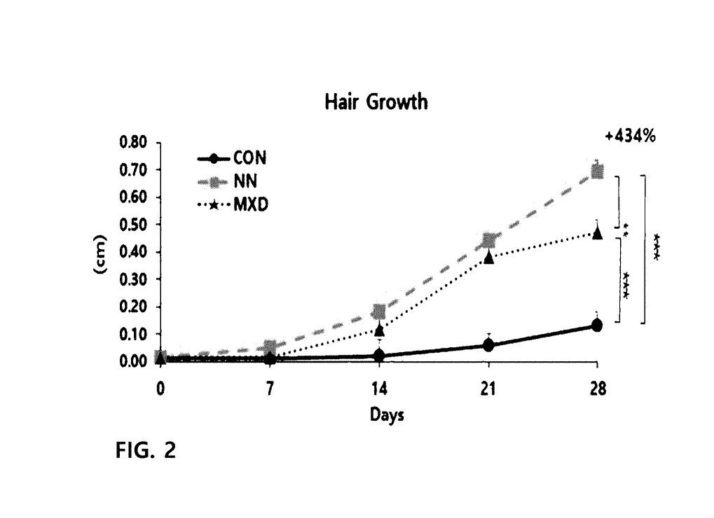

During the experimental period (4 weeks), a hair length was measured weekly. At three weeks, the hair length of the NN group was significantly increased, and after four weeks, the hair length was significantly increased by 434% (p<0.001), compared with the negative control group (FIG. 2). Therefore, it can be seen that nonanal has an excellent effect of promoting hair growth.

1-4. Histological Analysis of Dorsal Skin

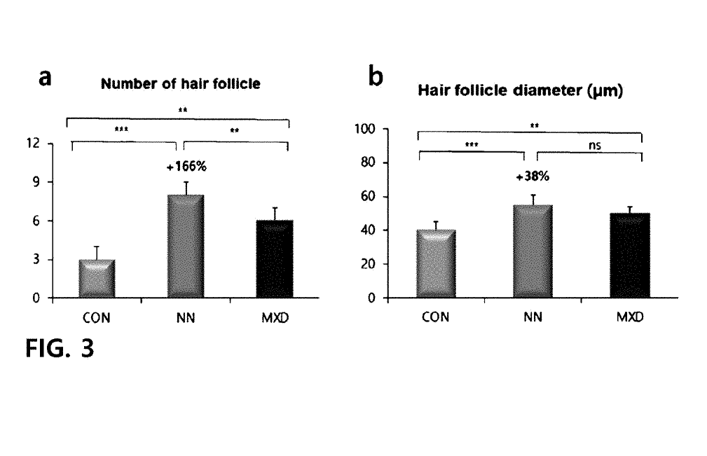

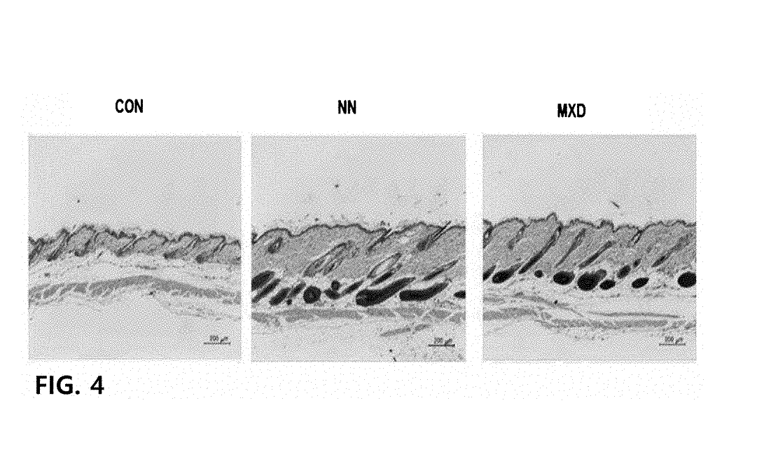

The experimental animals were sacrificed after four weeks of the application, and a dorsal skin tissue specimen was extracted from the test material-applied region using scissors and forceps, and then fixed with formalin. By steps, the resulting specimen was dehydrated with an alcohol and xylene, embedded in paraffin, sliced into a 5-μm fragment using a microtome, and treated again with an alcohol and xylene to remove paraffin. After hematoxylin-eosin staining, a histological change in hair follicle tissue was observed using an optical microscope. The hair development cycle was evaluated by a pathologist, the number and diameter of hair follicles were measured using an optical microscope, and the number of hair follicles in the test group was measured under a 40× optical microscope. The results are shown in FIGS. 3 and 4.

As a result, as shown in FIG. 3, in the NN group (166% increase) and the MXD group, compared with the negative control group (CON), the number of hair follicles was significantly increased, and as a result of evaluating the diameter of a hair follicle in each test group by image analysis, compared with the negative control group, hair follicle diameters were significantly increased in the NN group (38% increase) and the MXD group. In addition, as shown in FIG. 4, through microscopy, it was observed in the NN group and the MXD group that hair follicles elongated to come out of the skin, and it is considered that this phenomenon is related to a hair development-promoting effect of the two materials.

### Example 2

### Regulation of Hair Development-Related Gene and Protein Expression in Murine Skin Tissue by Nonanal

2-1. Analysis of Hair Development-Related Protein Expression in Skin Tissue

(1) Western Blot Analysis

A predetermined amount of skin tissue was homogenized with liquid nitrogen and a lysis buffer using a mortar and centrifuged at 13,000×g and 4° C. for 20 minutes to obtain an intermediate layer, and then a protein was quantified by the Bradford method. 50 μg of the protein was subjected to electrophoresis on an SDS polyacrylamide gel and electroblotted onto a PVDF hyperfilm, and then reacted with a β-catenin or β-actin antibody (Cell Signaling Technology, Danvers, Mass., USA). A signal of each protein was visualized using a chemiluminescent detection system (Amersham), and then a band thickness was quantified using the Quantity One Analysis Software (Bo-Rad Laboratories).

(2) Result of Analysis of Expression of Hair Development Promotion-Related Protein

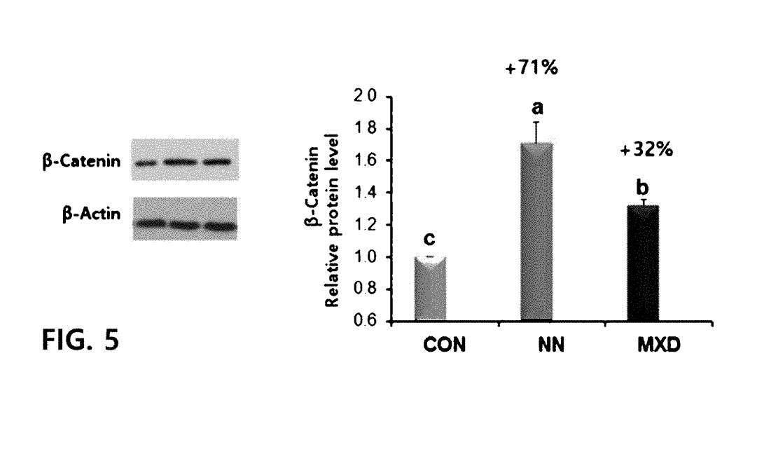

As a signaling system involved in regulating the hair follicle cycle and promoting entry into the anagen phase, a Wnt/β-catenin signaling system has been known. To confirm whether the hair development promotion-related signaling system was activated, as a result of analyzing a β-catenin expression level from dorsal skin tissue through Western blotting, compared with the negative control group, in the NN group, the β-catenin protein expression was significantly increased by 71%. Such an effect was superior to that of the Minoxidil group (FIG. 5).

2-2. Analysis of Hair Development Promotion-Related Gene Expression in Skin Tissue

(1) RNA Isolation Using TRIzol Method

To quantify total RNA in skin tissue of the experimental animals, 1 mL of a TRIzol solution was added to 50 to 100 mg of skin tissue and homogenized, and then centrifuged at 4° C. and 12,000×g for 10 minutes. The supernatant was transferred to a new tube, 200 μl of chloroform was added, and then the resulting mixture was vigorously shaken. This process was repeated twice, then the supernatant was transferred to a new tube, in which isopropanol and the supernatant were added in a ratio of 1:1. The resulting mixture was shaken 10 times, and maintained at room temperature for 10 minutes and centrifuged at 12,000×g and 4° C. for 10 minutes to remove the supernatant. Then, 1 ml of 70% ethanol was added to the remaining pellet and centrifuged at 7,500×g and 4° C. for 5 minutes. Following the removal of ethanol, a tube containing the RNA pellet was dried at room temperature for 5 minutes, and the RNA pellet was dissolved in nuclease-free water. Concentrations of the extracted RNA specimens at 260 nm and 280 nm wavelengths were measured using a UV/VIS spectrophotometer (Beckman Coulter, DU730), and agarose gel electrophoresis was performed to confirm the integrity of the RNA specimens.

(2) Quantitative-Polymerase Chain Reaction (q-PCR) Method

Following the isolation of RNA, cDNA was synthesized by performing reverse transcription using an oligo dT primer and a Superscript reverse transcriptase (Gibco BRL, Gaithersburg, Md., USA). To measure expression levels of genes, real-time q-PCR was performed using SYBR Green (Bio-Rad, Hercules, Calif., USA), and the instrument used was a CFX Connect™ Real-Time PCR Detection System (Bio-Rad). The base sequences of PCR primers for each gene are shown in Table 1 below.

For real time PCR, 2 μL of cDNA, 10 μL of a 2× SYBR mix, 1 μL each of a forward primer and a reverse primer (10 to 20 pmol/μL) were added, and then, H₂O was filled to the final volume 20 μL. A PCR extension step was performed as follows, and 35 extension cycles was performed. The extension was performed at 95° C. for 1 minute for hot start, at 95° C. for 15 seconds for denaturation in the extension step, at 55° C. for 30 seconds for annealing, and at 72° C. for 1 minute for extension. After all cycles were completed, to identify primer specificity, melting curve analysis was performed.

TABLE 1
Primer sequences used in q-PCR

| | | | Annealing |
|---|---|---|---|
| | | | temperature |
| Gene | Primer | Base sequence (5′→3′) | (° C.) |
| β-Catenin | F | ATGGCTACTCAAGCTGAC | 55 |
| | R | CAGCACTTTCAGCACTCTGC | |
| Cyclin D1 | F | TGAACTACCTGGACCGCT | 55 |
| | R | GCCTCTGGCATTTTGGAG | |
| Lymphoid enhancer | F | TGATTCCTGGTCCCCCTGGC | 58 |
| factor (LEF) | R | CACTGTCCGTGTGGGGGTGC | |
| T-Cell factor (TCF) | F | GTGTTACAGTGGCACGCTCTGACCCGT | 58 |
| | R | CCCTCACTCTCAGCGTCAGAC | |
| Glyceraldehyde-3- | F | AGAACATCATCCCTGCATCC | 55 |
| phosphatedehydrogenase | R | TCCACCACCCTGTTGCTGTA | |
| (GAPDH) | | | |

(3) Analysis Result for Regulation of Hair Development Promotion-Related Gene Expression

Change in expression of a β-catenin signaling material known as a mechanism for promoting hair development in skin tissue was analyzed by q-PCR analysis on dorsal skin tissue of a mouse to which the test material was applied for 4 weeks.

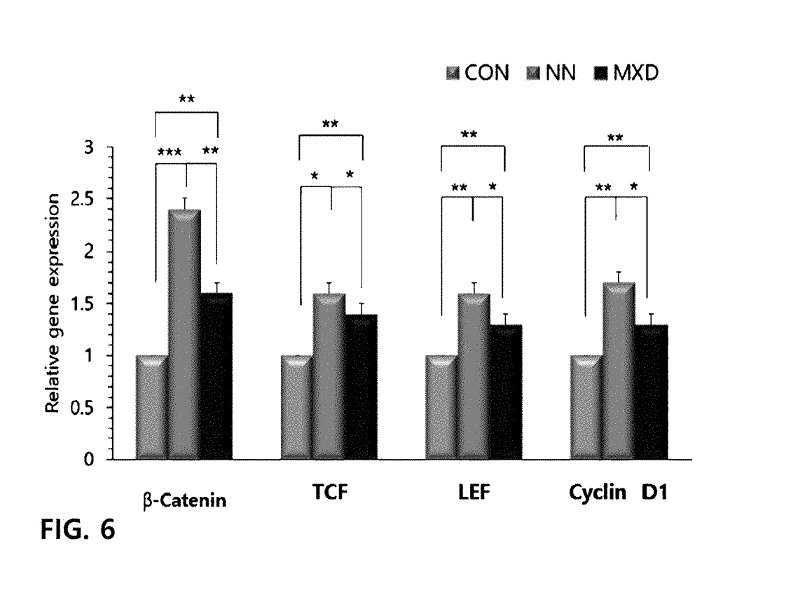

As a result, as shown in FIG. 6, in the NN group, it was observed that expression of β-catenin and downstream signaling transcription factors (TCF and LEF), and the target gene cyclin D1 was significantly increased, compared with the negative control group. Therefore, it can be seen that the effect of nonanal on promoting hair growth was exhibited by activation of β-catenin signaling.

What is claimed is:

1. A method for preventing or treating hair loss or promoting hair development or growth, the method comprising administering a composition including nonanal or a pharmaceutically acceptable salt thereof as an active ingredient, applying it or allowing it to be taken.

2. The method according to claim 1, wherein the composition is pharmaceutical composition, quasi-drug composition, cosmetic composition or health functional food composition.

3. The method according to claim 1, wherein the composition increases expression of a protein selected from the group consisting of
β-catenin, a T-cell factor (TCF), a lymphoid enhancer factor (LEF) and cyclin D1.

4. The method according to claim 2, wherein the pharmaceutical composition is prepared in the form selected from the group consisting of
a cream, a gel, a patch, a spray, an ointment, a plaster, a lotion, a liniment, a paste, and a cataplasm as a drug for topical use.

5. The method according to claim 2, wherein the cosmetic composition is prepared in the form of a scalp tonic, a scalp lotion, a scalp cream, a scalp serum, a scalp essence, a scalp ampoule, a scalp treatment, a scalp conditioner, a scalp shampoo, a scalp pack, a hair tonic, a hair lotion, a hair cream, a hair spray, a hair mousse, a hair gel, a hair conditioner, a hair shampoo, a hair rinse, a hair pack, a hair treatment, an eyebrow growth serum, an eyelash growth serum, an eyelash enhancer, and shampoo and rinse for pets.


SEQUENCE LISTING (10 sequences)

SEQ ID NO: 1 - DNA, 18 bp, Artificial
  beta-Catenin_F primer
atggctactcaagctgac

SEQ ID NO: 2 - DNA, 20 bp, Artificial
  beta-Catenin_R primer
cagcactttcagcactctgc

SEQ ID NO: 3 - DNA, 18 bp, Artificial
  Cyclin D1_F primer
tgaactacctggaccgct

SEQ ID NO: 4 - DNA, 18 bp, Artificial
  Cyclin D1_R primer
gcctctggcattttggag

SEQ ID NO: 5 - DNA, 20 bp, Artificial
  LEF_F primer
tgattcctggtccccctggc

SEQ ID NO: 6 - DNA, 20 bp, Artificial
  LEF_R primer
cactgtccgtgtgggggtgc

SEQ ID NO: 7 - DNA, 27 bp, Artificial
  TCF_F primer
gtgttacagtggcacgctctgacccgt

SEQ ID NO: 8 - DNA, 21 bp, Artificial
  TCF_R primer
ccctcactctcagcgtcagac

SEQ ID NO: 9 - DNA, 20 bp, Artificial
  GAPDH_F primer
agaacatcatccctgcatcc

SEQ ID NO: 10 - DNA, 20 bp, Artificial
  GAPDH_R primer
tccaccaccctgttgctgta